(19) United States
(12) Patent Application Publication
(10) Pub. No.: US 2017/0020669 A1
(43) Pub. Date: Jan. 26, 2017

(54) CATHETER SYSTEM FOR LOCATING AND IMPLANTING A REPLACEMENT BODY PART

(71) Applicant: BIOTRONIK AG, Buelach (CH)

(72) Inventors: Marc Bartels, Berlin (DE); Jens Ulmer, Kuesnacht (CH); Udo Kaempf, Berlin (DE)

(21) Appl. No.: 15/204,486

(22) Filed: Jul. 7, 2016

(30) Foreign Application Priority Data
Jul. 21, 2015 (DE) 10 2015 111 783.5

(51) Int. Cl. A61F 2/24

(52) U.S. Cl. CPC A61F 2/2433; A61F 2/2418

(57) ABSTRACT

A catheter system comprising at least one catheter shaft 3, an anisotropic implant, in particular an implantable heart valve prosthesis 6, a control device for controlling and manipulating the catheter, and at least three electrodes 4, 5 and 11 in the distal region of the catheter, which are each conductively connected to an analysis unit by their own electrode lead. The catheter system can change between an insertion state and an orientation state, wherein this change is triggered by a manipulation of the control device. In the orientation state the three electrodes of the catheter system span a spatial angle that is different from that spanned in the insertion state.

### CROSS REFERENCE TO RELATED APPLICATIONS

This application claims benefit of priority to German patent application DE 10 2015 111 783.5, filed Jul. 21, 2015; the entire content of which is herein incorporated by reference in its entirety.

### TECHNICAL FIELD

The present invention relates to medical devices and more specifically to a catheter system comprising at least one catheter shaft, an anisotropic implant, a control device for controlling and manipulating the catheter, and at least three electrodes in the distal region of the catheter, which are each conductively connected to an analysis unit via their own electrode lead.

### BACKGROUND OF THE INVENTION

Heart valves can be limited in terms of their functionality for various reasons. In the case of the aortic or mitral valve, both established aortic or mitral valve stenosis and an aortic or mitral valve insufficiency are possible. A valve stenosis can arise as a result of inflammation, but is more often the result of a progressive sclerosis (also referred to colloquially as hardening) of the valve, which leads to a degeneration and calcification of the valves and gradually to a stenosis. The stenosis, expressed more simply, leads to a constriction of the opening area of the valves, whereby an increasingly greater pressure is necessary in order to convey the same amount of blood through the valves. With continued progression of the disease, the heart ultimately reaches its limits and can no longer sufficiently convey blood; the patient is limited in terms of the activities they can perform.

Similar symptoms occur in the event of a valve insufficiency. In such cases the valves no longer close fully, resulting in a backflow of the conveyed blood. The physiological effect for the patient is similar: a sufficient amount of blood can no longer be conveyed via the damaged valve.

When the pumping capacity of the heart becomes too low on account of the valve damage, the damaged valves must be replaced. In the prior art there are essentially two different methods for this purpose.

The use of an artificial heart valve prosthesis as replacement of the aortic or mitral valve can be provided in an open, surgical procedure. An operation of this type is performed under general anaesthetic. The patient's chest is opened, the heart is separated from the blood circulation and the patient is provided with a blood supply via a heart-lung machine. The diseased aortic or mitral valve is then surgically removed and replaced by the implant, and lastly the heart is integrated back into the blood circulation, and the patient is disconnected from the heart-lung machine.

Recently, a catheter-based, minimally invasive procedure has become established as an alternative to the technique of open surgery. This new procedure is used particularly in older and weak patients, for whom a general anaesthetic poses a greater risk. The heart valve prostheses used for this purpose consist substantially of a basic structure and a valve arrangement secured therein.

The heart valve prosthesis is implanted via a catheter, which for example is inserted via the femoral artery. The heart valve prosthesis is then brought via this catheter to the site of implantation. There, the heart valve prosthesis is expanded and anchored in the vessel at the position of the natural heart valve. The natural heart valve in this case is not removed, but is merely displaced by the heart valve prosthesis.

For insertion by means of catheter, the heart valve prosthesis must be suitably mounted on the shaft of the catheter. Here, the diameter of the heart valve prosthesis must be much smaller for insertion than at the site of implantation. The heart valve prosthesis is accordingly compressed (“crimped”) onto the catheter shaft and expanded once it has been brought to the site of implantation. Here, the expansion can be performed, as is the case with a stent, in a self-expanding manner or by expansion of a balloon, depending on the basic structure.

A catheter of this type and a heart valve prosthesis of this type for the replacement of the natural aortic valve are described in EP 2 260 79, for example.

The mitral valve or also bicuspid valve (Valva atrioventricularis sinistra, Valva mitralis or Valva bicuspidalis) is located between the left atrium and left heart chamber (left ventricle), where it prevents the backflow of blood from the left heart chamber into the left atrium in the event of the contraction of the chamber. The form of the valve assimilates a mitre (bishop's headdress). The name bicuspid valve derives from the two cusps (or cuspides in Latin) that form this atrioventricular valve. The natural mitral valve does not have a rotationally symmetrical cross section, but an anisotropic cross section comparable to the letter D.

With the replacement of a mitral valve, a choice must therefore be made in principle between reproducing this anisotropic cross section in the heart valve prosthesis or using a rotationally symmetrical cross section. The use of a rotationally symmetrical cross section for the basic structure of a mitral valve prosthesis has the advantage that a basic structure of this type can be applied more easily to a rotationally symmetrical catheter. In addition, the mitral valve prosthesis “only” has to be brought by catheter to the correct location, i.e. the location of the natural mitral valve, and expanded. In the case of an implantation of this type there is no need to orientate the catheter and the mitral valve prosthesis with respect to the axis of rotation of the catheter or with respect to the anisotropic cross section of the natural mitral valve. The orientation of the angle of rotation of the axis of rotation or primary axis of the catheter with respect to an anisotropic environment such as the mitral valve annulus will also be referred to hereinafter as rotational orientation.

However, the implantation of a rotationally symmetrical mitral valve prosthesis into a genuine anisotropic environment is not optimal from a functional viewpoint or in terms of the physiological result of the prosthesis.

Accordingly, there remains a need for a system to improve the implantation of anisotropic implant

### SUMMARY OF THE INVENTION

The object of the present invention is therefore to develop a catheter system so that an anisotropic implant can be reliably implanted in an anisotropic environment. Here, the implant will be implanted under consideration of the orientation of the anisotropic implant with respect to the axis of rotation of the catheter system and with respect to the anisotropic conditions at the site of implantation.

The stated object is achieved by a catheter system having at least one catheter shaft, an anisotropic implant, a control device for controlling and manipulating the catheter, and at least three electrodes in a distal region of the catheter, which are each conductively connected to an analysis unit by a different electrode lead, characterised in that the catheter system changes between an insertion state and an orientation state, wherein the change is triggered by manipulating the control device, and wherein the at least three electrodes in the insertion state span a different spatial angle compared with a spatial angle spanned in the orientation state. Further advantageous embodiments of the invention are also disclosed herein.

The main concept of the invention is based on a combination of electrode catheter-based 3-dimensional mapping and navigation systems and catheter systems for implementation of heart valve prostheses, for example. It is essential to the invention that the catheter system can change between an insertion state and an orientation state.

Catheter systems having electrodes used for the diagnosis and ablation of cardiac arrhythmias are known in the prior art. Catheter systems of this type can be located and navigated in a patient using an accompanying 3-dimensional mapping and navigation system. An ablation catheter system of this type is disclosed by U.S. Pat. No. 6,050,267. Here, a catheter system is described which has an electrode for detection at the catheter tip, a proximal ring electrode, and a reference electrode for position determination. For 3-dimensional mapping and navigation, an additional 3 electrode pairs are adhered to the patient. These adhered electrodes pairs span a 3-dimensional coordinate system, along the axes of which the reference electrode of the catheter system is excited. By means of the interaction between excitation and reference electrode of the catheter system in combination with a system for 3-dimensional imaging (for example fluoroscopy), the catheter can be navigated through the patient in a controlled manner and an ablation can be performed at the desired location.

U.S. Pat. No. 8,241,274 discloses a similar catheter system comprising sensor navigation coils for the precise locating of a catheter system for implantation of a heart valve prosthesis in a 3-dimensional reference system.

However, the systems previously known in the prior art have the disadvantage described in the introduction that they do not provide any information relating to the rotational orientation of the axis of rotation of the catheter with respect to the reference system and therefore with respect to the patient.

The axis of rotation of the catheter within the scope of this application denotes the primary axis of the catheter. This is formed by the axis of symmetry of the tubular catheter shafts. The positional references ‘proximal’ and ‘distal’ within the scope of this application denote a position close to the practitioner and remote from the practitioner, respectively.

The axis of rotation or primary axis of the implant is understood to mean the primary axis of the main body. This is usually the longitudinal axis of the implant. In the case of a heart valve prosthesis, this is understood within the scope of the application by way of example to mean the primary axis of the main body of the heart valve prosthesis. The primary axis of the heart valve prosthesis coincides accordingly, in the event of correct implantation of the heart valve prosthesis, with the direction of flow of the blood through the heart valve prosthesis.

The catheter system according to the invention can change between an insertion state and an orientation state, wherein this change is triggered by a manipulation of the control device and at least three electrodes of the catheter system in the insertion state span a different spatial angle compared with that spanned in the orientation state.

As already mentioned, an anisotropic implant, in particular an anisotropic heart valve prosthesis, will be implanted suitably into anisotropic implantation environment with the aid of the catheter system according to the invention. The catheter system itself is substantially rotationally symmetrical, and merely the heart valve prosthesis arranged on a catheter shaft may have an anisotropy. A catheter system having intrinsically an easily detectable anisotropy would be difficult to produce and difficult to insert

An anisotropic implant is understood within the scope of the application to mean any implant of which the rotational state with respect to the environment at the site of implantation is not arbitrarily freely selectable.

The spanned spatial angle is determined by at least three electrodes. One electrode forms the apex of the angle; the other two electrodes form points that lie on the limbs of the spanned spatial angle. In the case of ring electrodes the points for determining the spanned spatial angle correspond to the centre point of the ring electrode. In the case of axially oriented electrodes the distal end points of the electrodes for example can be used to determine the spatial angle.

The invention is based on the concept of providing a catheter system having two states. The catheter system according to the invention can change between a rotationally symmetrical insertion state and an anisotropic orientation state. The detection of the orientation of the axis of rotation of the implant, in particular of the heart valve prosthesis, in 3-dimensional space is ensured in accordance with the invention in that the spatial angle spanned by at least 3 electrodes changes between insertion state and orientation state. The change between insertion state and orientation state and therefore the change of the spatial angle spanned by the electrodes is triggered by the control device.

The catheter system according to the invention enables not only the anatomically correct positioning of an anatomically adapted, anisotropic implant, in particular a heart valve prosthesis, but an exact positioning, which can be provided in a computer-assisted manner or in an entirely computer-controlled manner in cooperation with an accordingly designed 3-dimensional mapping and navigation system.

The system according to the invention is suitable in particular for implanting a heart valve prosthesis. This is true in particular for a heart valve prosthesis for replacing the natural mitral valve, having a main body with a D-shaped cross section as discussed further below.

The system according to the invention, however, is likewise advantageous in the case of implants such as occluder devices, stents for bifurcations, or intrinsically symmetrical implants having an artificial anisotropy. An implant having an artificial anisotropy could also be, for example, a heart valve prosthesis for the replacement of the natural aortic valve having an intrinsically rotationally symmetrical main body, in which a valve arrangement is secured. Here, the securing of the valve arrangement (for example by means of three sutures distributed over the circumference) can ensure an anisotropy, which must be adapted to the implantation environment (the natural aorta annulus or the exit of the coronary sinus arteries). The same is true when the rotational orientation of the valve arrangement is to be adapted to the orientation and natural position of the mitral or aortic valves.

The heart valve prosthesis is firstly guided together with the catheter system, similarly to the prior art, to the site of implantation, for example to the position of the natural mitral valve. Here, the catheter system is in the insertion state or basic state. When the position of the heart valve prosthesis axially in relation to the catheter axis coincides with the position of the natural heart valve, the orientation state of the catheter system with respect to the anisotropic natural heart valve of the patient is determined. The catheter system changes from the insertion state into the orientation state, the spatial angle spanned by at least 3 electrodes changes, and the orientation of the anisotropic heart valve prosthesis with respect to the anisotropic natural valve can be detected. Here, the change from the insertion state to the orientation state can be determined when the heart valve prosthesis is located at the site of implantation or proximally or distally thereof.

The change of the spatial angle can be detected by 3-dimensional mapping systems, as are known in the prior art, for example in U.S. Pat. No. 6,050,267. Since the positioning of the anisotropic heart valve prosthesis and the change of the spatial angle are fixed and known, the orientation of the anisotropic heart valve prosthesis with respect to the anisotropic implantation environment can be directly determined via the detection of the spatial angle change. The electrodes provided in the catheter system according to the invention enable, in conjunction with the 3-dimensional mapping system (for example by means of 3 pairs of different reference electrodes, which are attached to the patient), the detection of the change of the electric dipole in the reference system. By means of the predefined relationship between insertion state and orientation state, the orientation of the catheter system or the orientation of the axis of rotation of the catheter system can be directly determined from the change of the spatial orientation of the electric dipole. The asymmetric design and arrangement of the at least three electrodes makes it possible for the first time to detect a spatial arrangement thereof on the basis of a measurement. The system functions similarly with the use of a magnetic field instead of the adhered electrode pairs as reference system.

The change between insertion state and orientation state is preferably reversible and can be repeated as often as desired.

In the orientation state at least one electrode is preferably no longer arranged on the primary axis of the catheter system in the insertion state. In this preferred embodiment the catheter system has three electrodes, which are arranged from proximally to distally and are spaced apart from one another. In the insertion state all 3 electrodes are located on the axis of rotation and primary axis of the catheter system. In the orientation state one of these electrodes is moved away from the primary axis of the catheter system. By means of the known relationship between the direction of movement of the electrode from the insertion state into the orientation state and the orientation of the anisotropic implant, in particular the heart valve prosthesis, with respect to the primary axis of the catheter and the direction of movement, the orientation of the primary axis of the catheter system or the primary axis of the implant, in particular the heart valve prosthesis, in the reference system and that therefore in the patient can thus be unambiguously determined. In the simplest case, for example, part of the catheter shaft having at least one electrode is bent with respect to the rest of the catheter shaft having the rest of the electrodes in order to change from the insertion state into the orientation state. This bending is performed at a defined angle and in a defined direction with respect to the anisotropic implant, in particular the anisotropic heart valve prosthesis. The spatial angle spanned by at least three electrodes changes accordingly in a defined manner.

In an advantageous embodiment of the invention the electrodes are embodied as ring electrodes and the electrode leads are embedded in a catheter shaft. However, individual lumina for the electrode leads would also be expedient. A ring electrode within the scope of the invention is understood to mean an electrical conductor that is arranged annularly around the primary axis of the catheter system, preferably on a catheter shaft. More simply, a ring electrode is therefore a ring made of electrically conductive material around a catheter shaft. An electrode lead is understood to mean the corresponding point of electrical contact of the electrode with a possible voltage source or analysis/control unit proximally and outside the patient in the case of correct use of the catheter system. Electrode arrangements of this type can be particularly easily transferred from an insertion state into an orientation state. However, one of the electrodes can advantageously also be embodied as a distal electrode at the distal end of the catheter system (what is known as a tip electrode).

In one embodiment at least two electrodes are arranged on two different catheter shafts, of which the main axes extend in parallel in the insertion state and which are preferably fixedly connected to one another at least in part. In this embodiment of the invention the catheter system has at least two catheter shafts, which are connected to one another at least in part and of which the main axes are both parallel to one another and parallel to the primary axis of the catheter system in the insertion state thereof In this embodiment of the invention the change between insertion state and orientation state of the catheter system is performed in that the two catheter shafts each having at least one electrode are moved relative to one another in a defined manner. Here as well, again in the simplest embodiment, one catheter shaft can be bent relative to the other catheter shaft, whereby the spatial angle comprised on the whole by the electrodes changes.

In the case of a system of this type having parallel catheter shafts, these can in turn additionally also have a plurality of catheter shafts arranged inside one another, which are movable relative to one another.

The heart valve prosthesis expediently has a basic structure and a valve arrangement, wherein the basic structure consists of a self-expanding or balloon-expandable material and the basic structure preferably has an anisotropic, particularly preferably a D-shaped cross section. The basic structure is in this embodiment of the invention expanded at the site of implantation from its compressed form in the insertion state and is thus anchored at the location of the natural valve. Here, the natural heart valves are usually displaced by the basic structure. However, a minimally invasive removal of the natural valves is also conceivable. The valve arrangement is fixed in the basic structure and is anchored via this at the location of the natural valve. The valve arrangement takes over the valve function of the natural valve and can accordingly change between an open and a closed state with respect to the natural direction of the blood flow.

The change between compressed state and expanded, implanted state of the heart valve prosthesis can be implemented here either via the inflation of a balloon, or by the basic structure being formed from self-expanding material. In this case the basic structure is held in its compressed state by means of a mechanical force (for example a case covering the heart valve prosthesis). After removing the holding force (for example by proximally retracting the case covering the heart valve prosthesis), the basic structure and therefore the heart valve prosthesis automatically expands and is anchored at the location of the natural valve.

In a further preferred embodiment a first catheter shaft has a lumen for a guide wire and carries the implant, in particular the heart valve prosthesis, whereas a second catheter shaft surrounds the first catheter shaft and with its distal region covers the implant, in particular the heart valve prosthesis. This embodiment is considered especially for self-expanding implants, in particular a heart valve prosthesis, having a self-expanding basic structure. The distal part of the second catheter shaft (often referred to as a case in the prior art) surrounds the heart valve prosthesis and holds this in the compressed state during the insertion state. In the insertion state the catheter system is advanced in the patient until the heart valve prosthesis is located at the location of the natural heart valve. The catheter system now changes from its insertion state to its orientation state, and the rotational orientation of the primary axis of the catheter system and therefore the rotational orientation of the primary axis of the heart valve prosthesis is determined. If the rotational orientation of the anisotropic heart valve prosthesis coincides with the anisotropic implantation environment, the second catheter shaft is retracted proximally and the heart valve prosthesis is released. The basic structure of the heart valve prosthesis expands and is thus anchored at the site of implantation.

In another embodiment a first catheter shaft has a lumen for a guide wire and a second catheter shaft surrounds the first catheter shaft, wherein the second catheter shaft has a lumen for a fluid and an inflatable balloon at its distal end, wherein the implant, in particular the heart valve prosthesis, is arranged over the balloon and can be expanded by means is of inflation of the balloon. This embodiment functions, with respect to the determination of the rotational orientation, in exactly the same way as the previously described embodiment. However, the basic structure of the implant or the heart valve prosthesis is in this embodiment expanded and anchored at the site of implantation by means of inflation of a balloon located beneath said implant or heart valve prosthesis. Fluid is applied via the lumen of the second catheter shaft provided for this purpose.

The heart valve prosthesis is particularly preferably suitable for implantation at the location of the mitral valve. The advantages of the invention are particularly apparent in the case of a heart valve prosthesis having an anisotropic basic structure, in particular a basic structure having a D-shaped cross section, for replacement of the natural mitral valve. The heart valve prosthesis is particularly preferably therefore embodied as a mitral valve prosthesis having a basic structure, wherein the basic structure has a D-shaped cross section. A D-shaped cross section is understood within the scope of this application to mean a cross section which has an approximately straight portion and a curved (preferably circularly or elliptically) portion.

The control device is preferably designed for the automatic control of the catheter system in cooperation with a 3-D mapping system of a patient. In this embodiment of the invention the heart valve prosthesis is implanted automatically by means of computer control. Here, the catheter system and the heart valve prosthesis arranged thereon is not only guided to the site of implantation in a computer-assisted manner, but also automatically changes between insertion state and orientation state. Depending on the determined rotational orientation of the catheter system/the heart valve prosthesis, the rotational orientation is corrected and the heart valve prosthesis is expanded and implanted at the site of implantation.

The present invention makes it possible to implant in particular a (anisotropic) heart valve prosthesis at the target site with accurate rotational orientation. The catheter system has all prerequisites for computer-assisted, automatic implantation.

### BRIEF DESCRIPTION OF THE DRAWINGS

FIG. 1a is a schematic of a first embodiment of the catheter system shown in an insertion state and FIG. 1b is a schematic of the first embodiment shown in an orientation state.

FIG. 2a is a schematic of a second embodiment of the catheter system shown in an insertion state and FIG. 2b is a schematic of the second embodiment shown in an orientation state.

FIG. 3a is a schematic of a third embodiment of the catheter system shown in an insertion state and FIG. 3b is a schematic of the third embodiment shown in an orientation state.

FIG. 4a is a schematic of a fourth embodiment of the catheter system shown in an insertion state and FIG. 4b is a schematic of the fourth embodiment shown in an orientation state.

FIG. 5a is a schematic of a fifth embodiment of the catheter system shown in an insertion state and FIG. 5b is a schematic of the fifth embodiment shown in an orientation state.

FIG. 6a is a schematic of a sixth embodiment of the catheter system shown in an insertion state and FIG. 6b is a schematic of the sixth embodiment shown in an orientation state.

### DETAILED DESCRIPTION OF PREFERRED EMBODIMENTS

The invention will be described hereinafter on the basis of an implantable heart valve prosthesis for replacement of the natural mitral valve, but is not limited to this. The invention relates to any catheter system comprising an anisotropic implant as described in the introduction. These can be implants such as occluder devices, stents for bifurcations, or intrinsically symmetrical implants having an artificial anisotropy (such as a marker). What is key is that the implant has an anisotropy. According to this application the anisotropy of the implant is present with respect to the axis of rotation or primary axis of the implant. The primary axis of the implant is usually the longitudinal axis. If the implant is in the configuration for insertion (insertion state), the primary axis usually corresponds to the catheter axis.

The invention will be described in greater detail hereinafter on the basis of the exemplary embodiments illustrated in the drawings.

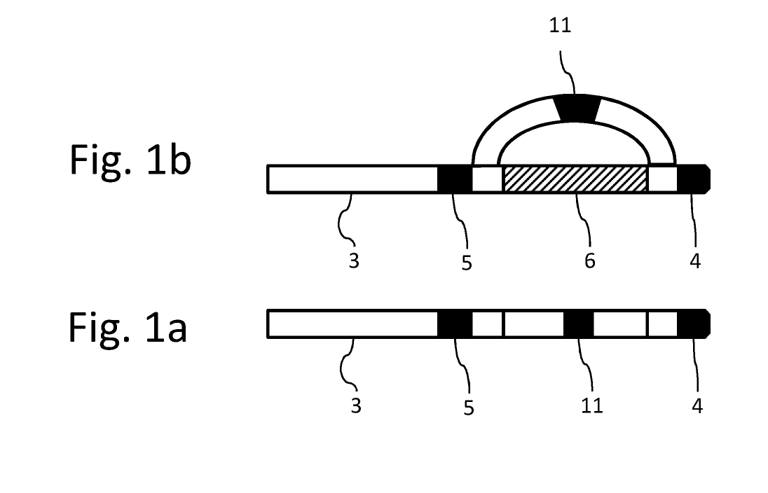

FIGS. 1 to 6 schematically illustrate six exemplary embodiments of a catheter system in the sense of the present invention. Here, FIGS. 1b, 2b, 3b, 4b, 5b and 6b show the orientation state of the catheter system, whereas FIGS. 1a, 2a, 3a, 4a, 5a and 6a describe the insertion state of the catheter system. Like components have the same reference signs in all drawings.

FIG. 1 schematically shows a first exemplary embodiment of a catheter system according to the invention in its insertion state (FIG. 1a) and its orientation state (FIG. 1b). The catheter system has two different catheter sheaths, of which the main axes extend substantially in parallel and are connected to one another at least in part. In the insertion state (FIG. 1a) only one catheter shaft 3 is essentially illustrated, which has a proximal ring electrode 5 and an orientation electrode 11, which is arranged distally thereof and is likewise embodied as a ring electrode. The second catheter shaft, which is arranged in parallel with and is connected to the first shaft, carries the heart valve prosthesis, for example a mitral valve prosthesis 6, and a distal ring electrode 4.

In the insertion state the main axes of the two catheter shafts are parallel to the primary axis of the catheter system. In the orientation state (FIG. 1b) the orientation electrode 11 is deflected by an arcuate curvature of the catheter shaft. The spatial angle spanned by the proximal ring electrode 5, the orientation electrode 11 and the distal ring electrode 4 changes accordingly. The rotational orientation of the heart valve prosthesis 6 can be determined from this change of the spanned spatial angle via a corresponding reference system (not illustrated). Following appropriate adaptation of the rotational orientation, the heart valve prosthesis 6 can be expanded, for example by inflation of a balloon, and can be implanted.

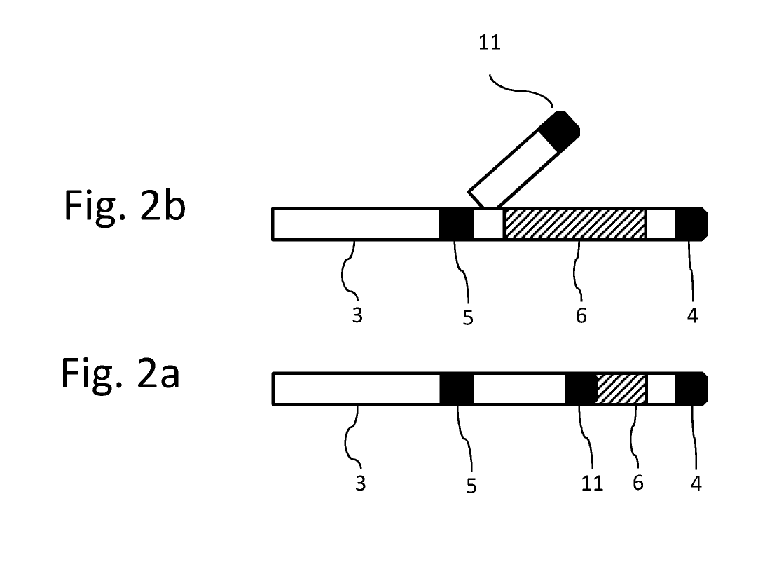

FIG. 2 likewise schematically shows an exemplary embodiment of the invention, wherein here the catheter shaft 3 with the orientation electrode 11 is shorter than the catheter shaft carrying the heart valve prosthesis 6 and the distal ring electrode 4. In the orientation state (FIG. 2b) the distal end of the catheter shaft 3 with the orientation electrode 11 is bent.

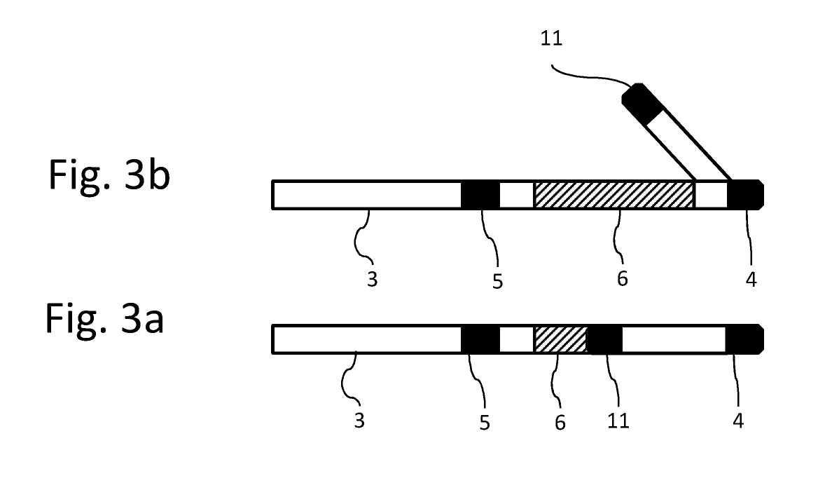

The exemplary embodiment in FIG. 3 differs from the exemplary embodiment according to FIG. 2 in that the orientation electrode 11 is bent from the distal end of the catheter shaft.

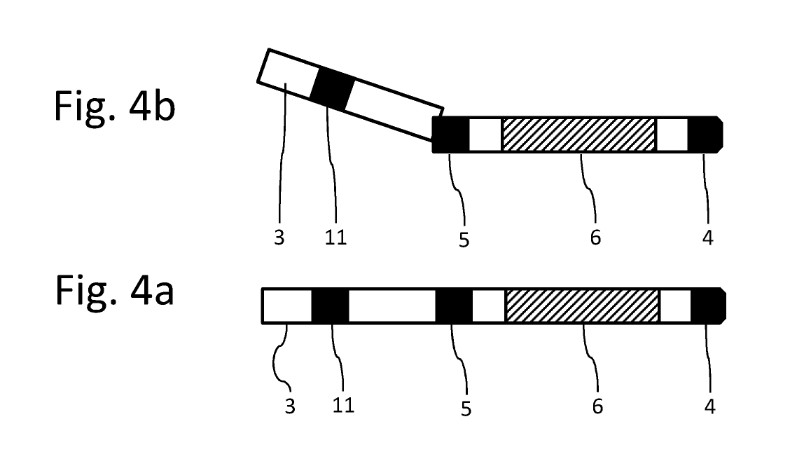

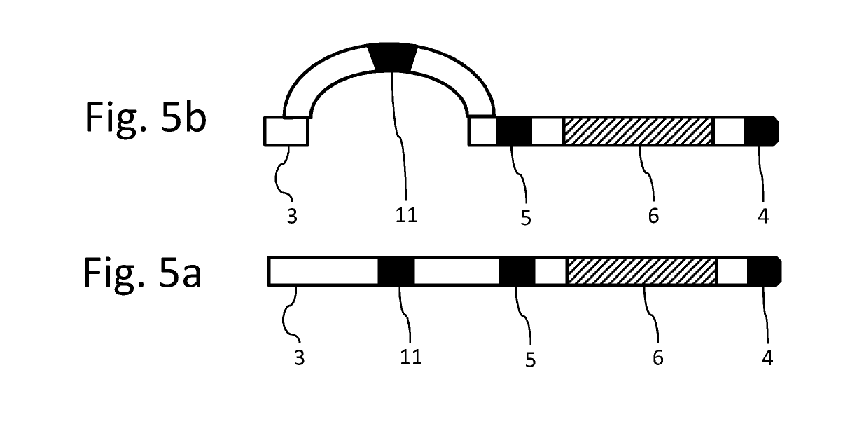

The exemplary embodiments of FIGS. 4 to 6 are exemplary embodiments without catheter shafts arranged in parallel. In the exemplary embodiment according to FIGS. 4 and 5 the orientation electrode 11 is located proximally of the ring electrodes 5 and 4. Here, a change of the spatial angle spanned by the orientation electrode 11, the proximal ring electrode 5 and the distal ring electrode 4 is achieved by a bending (FIG. 4b) or kinking (FIG. 5b) of the proximal end of the catheter shaft 3.

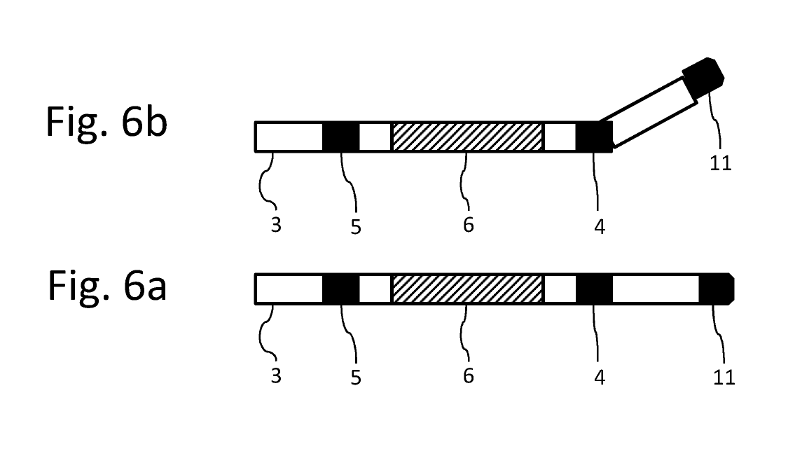

FIG. 6 shows an exemplary embodiment of a catheter system having a self-expanding heart valve prosthesis. The heart valve prosthesis 6 is surrounded by a second catheter shaft 3 and is held by this in the compressed state in the insertion state (FIG. 6a). Two ring electrodes are arranged proximally (ring electrode 5) and distally (ring electrode 4) of the heart valve prosthesis. When changing from the insertion state (FIG. 6a) into the orientation state (FIG. 6b) the entire catheter system with catheter shaft 3 and inner shaft (first catheter shaft, which carries the heart valve prosthesis) arranged therein bends, or only the inner shaft distally of the heart valve prosthesis and the distal ring electrode 4 bends. When only the inner shaft bends, the outer shaft is expediently formed shorter than the inner shaft. By bending the distal end comprising the orientation electrode 11, the spatial angle spanned by ring electrode 5, ring electrode 4 and orientation electrode changes. Once the rotational orientation of the heart valve prosthesis 6 has been set in the orientation state, this is left and the heart valve prosthesis 6 is released and implanted by retracting the outer shaft 3.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A catheter system comprising at least one catheter shaft, an anisotropic implant (6), a control device for controlling and manipulating the catheter, and at least three electrodes (4, 5, 11) in a distal region of the catheter, which are each conductively connected to an analysis unit by a different electrode lead, characterised in that the catheter system changes between an insertion state and an orientation state, wherein the change is triggered by manipulating the control device, and wherein the at least three electrodes (4, 5, 11) in the insertion state span a different spatial angle compared with a spatial angle spanned in the orientation state.

2. The catheter system according to claim 1, characterised in that in the orientation state at least one electrode (11) is no longer arranged on a primary axis of the catheter system in the insertion state.

3. The catheter system according to claim 1, characterised in that the electrodes (4, 5, 11) are embodied as ring electrodes and the electrode leads are embedded in a catheter shaft.

4. The catheter system according to claim 1, characterised in that at least two electrodes are arranged on two different catheter shafts, of which the main axes extend in parallel in the insertion state and which are optionally fixedly connected to one another at least in part.

5. The catheter system according to claim 1, characterised in that the control device is designed for the automatic control of the catheter system in cooperation with a 3-D mapping system of a patient.

6. The catheter system according to claim 1, characterised in that the anisotropic implant is formed as a heart valve prosthesis (6).

7. The catheter system according to claim 6, characterised in that the heart valve prosthesis (6) has a basic structure and a valve arrangement, wherein the basic structure consists essentially of a self-expanding or balloon-expandable material and optionally has an anisotropic, optionally D-shaped cross section.

8. The catheter system according to claim 6, characterised in that a first catheter shaft has a lumen for a guide wire and carries the heart valve prosthesis (6), and a second catheter shaft surrounds the first catheter shaft and with its distal region covers the heart valve prosthesis.

9. The catheter system according to claim 6, characterised in that a first catheter shaft has a lumen for a guide wire and a second catheter shaft surrounds the first catheter shaft, wherein the second catheter shaft has a lumen for a fluid and an inflatable balloon at its distal end in communication with the fluid, wherein the heart valve prosthesis (6) is arranged over the balloon and can be expanded by means of inflation of the balloon.

10. The catheter system according to claim 6, characterised in that the heart valve prosthesis (6) is suitable for implantation at the location of the mitral valve.
